(19) United States
(12) Patent Application Publication
(10) Pub. No.: US 2024/0310380 A1
(43) Pub. Date: Sep. 19, 2024

(54) EXON 1 FRAMESHIFT ANTIGENS FOR VACCINES, THERAPEUTICS, AND DIAGNOSTICS

(71) Applicant: Arizona Board of Regents on behalf of Arizona State University, Scottsdale, AZ (US)

(72) Inventor: Stephen Albert Johnston, Tempe, AZ (US)

(21) Appl. No.: 18/678,311

(22) Filed: May 30, 2024

Related U.S. Application Data
(63) Continuation of application No. PCT/US2022/081027, filed on Dec. 6, 2022.
(60) Provisional application No. 63/287,422, filed on Dec. 8, 2021.

(51) Int. Cl. G01N 33/574; A61K 39/00

(52) U.S. Cl. CPC G01N 33/57488; A61K 39/0011; A61K 2039/585

(57) ABSTRACT

Provided herein are methods of identifying neoantigens for diagnosing, treating, and preventing cancer. Also provided herein are methods of measuring antibody response to one or more frameshift peptides caused by exon 1 mis-initiation. Also disclosed are methods and compositions for administering identified neoantigens for the treatment and prevention of cancer.

### INCORPORATION BY REFERENCE TO ANY PRIORITY APPLICATIONS

The present application is a continuation of PCT Application No. PCT/US2022/081027, filed Dec. 6, 2022, which claims the benefit of U.S. Provisional Patent Application Ser. No. 63/287,422, filed Dec. 8, 2021, each of which is incorporated herein by reference in its entirety.

### REFERENCE TO SEQUENCE LISTING

The present application is being filed along with a sequence listing in electronic format. The sequence listing is provided as a file entitled “SEQLISTING_CALV034C1.xml”, created May 29, 2024, which is 16.7 MB in size. The information in the electronic format of the sequence listing is incorporated herein by reference in its entirety.

### BACKGROUND

Checkpoint inhibitor immunotherapeutics are revolutionizing cancer therapy. However, even in the most responsive cancers a substantial portion (50%-80%) of the patients have poor to no positive response. The evidence to date is that whether a patient has an effective response to the treatment depends on the nature of the immune response they have established against the tumor. More specifically, the level and quality of the immune response to neoantigens in the cancer seems to be most important.

### SUMMARY

Provided herein, in certain aspects, are arrays and compositions, and methods of making and using the arrays and compositions for diagnostic, preventative and therapeutic purposes.

Accordingly, some embodiments provided herein are arrays. In some embodiments, the arrays include frameshift peptides (FSPs) resulting from exon 1 mis-initiation of translation. In some embodiments, the FSPs on the array are spaced between 3 and 9 μm, such as 3, 4, 5, 6, 7, 8, or 9 μm, or an amount within a range defined by any two of the aforementioned values. In some embodiments, the array is used to diagnose cancer. In some embodiments, the arrays are used to predict response to immunotherapy. In some embodiments, the arrays are used to predict adverse responses to immunotherapy. In some embodiments, the FSPs are used to design therapeutic or prophylactic vaccines. In some embodiments, the FSPs comprise one or more peptides as set forth in SEQ ID NOs: 1-19,997. In some embodiments, the arrays include at least about 2500, about 5000, about 7500, about 10000, about 12500, about 15000, about 17500, about 20000, about 22500, about 25000, about 27500, about 30000, about 32500, about 35000, about 37500, about 40000, about 50000, about 100000, about 200000, about 300000, or about 400000 FSPs, or an amount within a range defined by any two of the aforementioned values.

Some embodiments provided herein relate to vaccine compositions. In some embodiments, biological samples, such as blood, from cancer patients are applied to the FSP arrays described herein to determine reactivity of peptides for each patient. In some embodiments, FSPs unique to the patient are used in a personal vaccine. In some embodiments, FSPs shared between different patients are used for off-the-shelf therapeutic or preventative vaccines. In some embodiments, the vaccine compositions include frameshift peptides (FSPs) resulting from exon 1 mis-initiation of translation. In some embodiments, the vaccine compositions further include an adjuvant. In some embodiments, the adjuvant is ABM2, AS01B, AS02, AS02A, Adjumer, Adjuvax, Algammulin, Alum, Aluminum phosphate, Aluminum potassium sulfate, Bordetella pertussis, Calcitriol, Chitosan, Cholera toxin, CpG, Dibutyl phthalate, Dimethyldioctadecylammonium bromide (DDA), Freund's adjuvant, Freund's complete, Freund's incomplete (IFA), GM-CSF, GMDP, Gamma Inulin, Glycerol, HBSS (Hank's Balanced Salt Solution), polyinosinic-polycytidylic acid stabilized with polylysine and carboxymethylcellulose (poly-ICLC, also known as Hiltonol), IL-12, IL-2, Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non-ionic copolymer), Pam3Cys (lipoprotein), Pertussis toxin, Poloxamer, QS21, RaLPS, Ribi, Saponin, Seppic ISA 720, Soybean Oil, Squalene, Syntex Adjuvant Formulation (SAF), Synthetic polynucleotides (poly IC/poly AU), TiterMax Tomatine, Vaxfectin, XtendIII, or Zymosan.

Some embodiments provided herein relate to methods of detecting cancer in a subject. In some embodiments, the methods include contacting a biological sample from the subject to an array comprising frameshift peptides (FSPs) resulting from exon 1 mis-initiation of translation and measuring antibody reactivity to the FSPs resulting from mis-initiation of translation in tumors. In some embodiments, the FSPs comprise one or more peptides as set forth in SEQ ID NOs: 1-19,997. In some embodiments, the FSPs are fixed on a substrate. In some embodiments, the substrate comprises glass, silica, composite, resin, or combination thereof. In some embodiments, the array is configured to detect binding by at least one of fluorescence, luminescence, calorimetry, chromatography, radioactivity, Bio-Layer Interferometry, and surface plasmon resonance. In some embodiments, the array comprises at least about 2500, about 5000, about 7500, about 10000, about 12500, about 15000, about 17500, about 20000, about 22500, about 25000, about 27500, about 30000, about 32500, about 35000, about 37500, about 40000, about 50000, about 100000, about 200000, about 300000, or about 400000 FSPs, or an amount within a range defined by any two of the aforementioned values. In some embodiments, the biological sample comprises blood, serum, plasma, cerebrospinal fluid, saliva, urine, or combinations thereof. In some embodiments, the biological sample comprises an antibody. In some embodiments, the subject is a mammal. In some embodiments, the subject is a human, a dog, a cat, a mouse, a rat, a rabbit, a horse, a cow, or a pig. In some embodiments, the subject is suspected of having a cancer. In some embodiments, the cancer is selected from the group consisting of Acute lymphoblastic leukemia, Acute monocytic leukemia, Acute myeloid leukemia, Acute promyelocytic leukemia, Adenocarcinoma, Adult T-cell leukemia, Astrocytoma, Bladder cancer, Bone Cancer, Brain Tumor, Breast Cancer, Burkitt's lymphoma, Carcinoma, Cervical Cancer, Chronic Lymphocytic Leukemia, Chronic myelogenous leukemia, Colon Cancer, Colorectal cancer, Endometrial cancer, Glioblastoma multiforme, Glioma, Hepatocellular carcinoma, Hodgkin's lymphoma, Inflammatory breast cancer, Kidney Cancer, Leukemia, Lung cancer, Lymphoma, Malignant Mesothelioma, Medulloblastoma, Melanoma, Multiple myeloma, Neuroblastoma, Non-Hodgkin Lymphoma, Non-Small Cell Lung Cancer, Ovarian Cancer, Pancreatic Cancer, Pituitary tumor, Prostate cancer, Retinoblastoma, Skin Cancer, Small Cell Lung Cancer, Squamous cell carcinoma, Stomach cancer, T-cell leukemia, T-cell lymphoma, Thyroid cancer, and Wilms' tumor.

Some embodiments provided herein relate to methods of treating or preventing cancer. In some embodiments, the methods include administering a therapeutic molecule designed to bind frameshift peptides (FSPs) resulting from exon 1 mis-initiation of translation. In some embodiments, the therapeutic molecule is an antibody or synthetic antibody. In some embodiments, the therapeutic molecule binds a FSP resulting from exon 1 mis-initiation of translation. In some embodiments, the therapeutic molecule is a vaccine. In some embodiments, the vaccine comprises FSPs resulting from exon 1 mis-initiation of translation. In some embodiments, the FSPs comprise one or more peptides as set forth in SEQ ID NOs: 1-19,997.

Some embodiments provided herein relate to methods of predicting response to an immunotherapy. In some embodiments, the methods include identifying frameshift peptides (FSPs) resulting from exon 1 mis-initiation of translation. In some embodiments, the methods are used to predict adverse immune responses to immunotherapy.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1: shows a model for RNA based frame-shift peptide (FSP) production in normal cells versus cancer cells.

FIG. 2: shows a model of normal initiation versus mis-initiation at exon 1, creating FSPs.

FIG. 3: shows how FSPs predicted to be produced by mis-initiation of translation of exon 1 can be screened for those with clinical utility for vaccines, therapeutics, or diagnostics.

FIG. 4A: shows common reactivity and cancer-type reactivity against FS peptides were represented by ˜7000 selected FS peptides. PC: pancreatic cancer; LC: lung cancer; GBM: glioblastoma; GC: gastric cancer; and BC: breast cancer; (n=17/each cancer type) and a set of non-cancer samples (n=64), as control. A subset of these peptides is from exon 1 mis-initiations.

FIG. 4B: shows p-value and fold change volcano plot analysis of cancer IgG reactivity on the 400K FS array compared to normal. The horizontal line represents the significant p-value cut-off= 1/392328 (the number of the array peptides). A subset of these significant peptides is from exon 1 mis-initiations.

FIG. 4C: shows a distribution of personal anti-FS response and shared anti-FS response in all 5 cancer types. A subset of these significant peptides is from exon 1 mis-initiations.

FIG. 4D: shows components of cancer-type specific FS vaccines, top 100 FS peptides for each cancer type were selected with highest positive rate in corresponding cancer type. Shading in normal represents a negative sample; other shading is indicative of a positive sample. A subset of these significant peptides is from exon 1 mis-initiations.

FIG. 4E: shows components of a general FS vaccine, top 100 FS peptides were selected with highest positive rate in cancer group. Shading in normal represents a negative sample; other shading is indicative of a positive sample. A subset of these significant peptides is from exon 1 mis-initiations.

FIG. 4F: shows a heat map of the positive rate distribution of the FS peptides in Stage I and late stages pancreatic cancer. A subset of these significant peptides is from exon 1 mis-initiations.

FIG. 5: shows that exon 1 FSPs are useful in diagnosing stage 1 breast cancer and in creating a potential vaccine for breast cancer.

FIG. 6: shows that exon 1 FSPs are predictive of a subject's response to immunotherapeutic treatment of lung cancer. Shown are the performance results of a model using 226 FSPs, 32 of these were FSPs derived from exon 1 mis-initiation errors. which shows performance of the response to treatment model compared to observed response outcomes. The ordered-scores map displays observed or predicted non-progressors (bottom) and observed or predicted progressors (top). Within the 226-peptide classifying model, 32 peptides were generated from exon 1 mis-initiations.

### DETAILED DESCRIPTION

Throughout the description, including in the Summary and Drawings, reference is made to particular features of the disclosure. It is to be understood that the disclosure in this specification includes all possible combinations of such particular features. For example, where a particular feature is disclosed in the context of a particular aspect or embodiment of the disclosure, or a particular claim, that feature can also be used, to the extent possible, in combination with and/or in the context of other particular aspects and embodiments of the disclosure, and in the disclosure generally.

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there is a plurality of definitions for a term herein, those in this section prevail unless stated otherwise.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least.” When used in the context of a process, the term “comprising” means that the process includes at least the recited steps but may include additional steps. When used in the context of a compound, composition or device, the term “comprising” means that the compound, composition, or device includes at least the recited features or components, but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to an advantage. Any reference signs in the claims should not be construed as limiting the scope.

Provided herein are methods and compositions for preventing, treating, and diagnosing cancer comprising the use of neoantigens. Neoantigens herein comprise peptides encoded by nucleic acids having frameshift mutations, in particular relevant here are FSPs created by mis-initiation of exon 1, causing a frameshift in the mRNA and a long stretch of non-natural amino acids that are, in some cases, recognized as a non-self peptide by the immune system.

The success of checkpoint inhibitors in cancer therapy is largely attributed to activating the patient's immune response to their tumor's neoantigens arising from DNA mutations. This realization has motivated the interest in personal cancer vaccines based on sequencing the patient's tumor DNA to discover neoantigens. Embodiments provided herein relate to an additional, unrecognized source of tumor neoantigens. In some embodiments, errors in transcription of microsatellites (MS) and mis-splicing of exons create highly immunogenic frameshift (FS) neoantigens in tumors. Another source is the frameshift peptides (FSPs) produced by mis-initiation of translation of exon 1. The sequence of these FS neoantigens are bioinformatically predictable, allowing creation of a peptide array representing all possible neoantigen FS peptides. This array can be used to detect the antibody response in a patient to the FS peptides. A survey of 5 types of cancers revealed peptides that are personally reactive for each patient and those that are shared between individuals and even across different tumor types. This source of neoantigens and the method to discover them may be useful in developing cancer vaccines, diagnostics, and therapeutics.

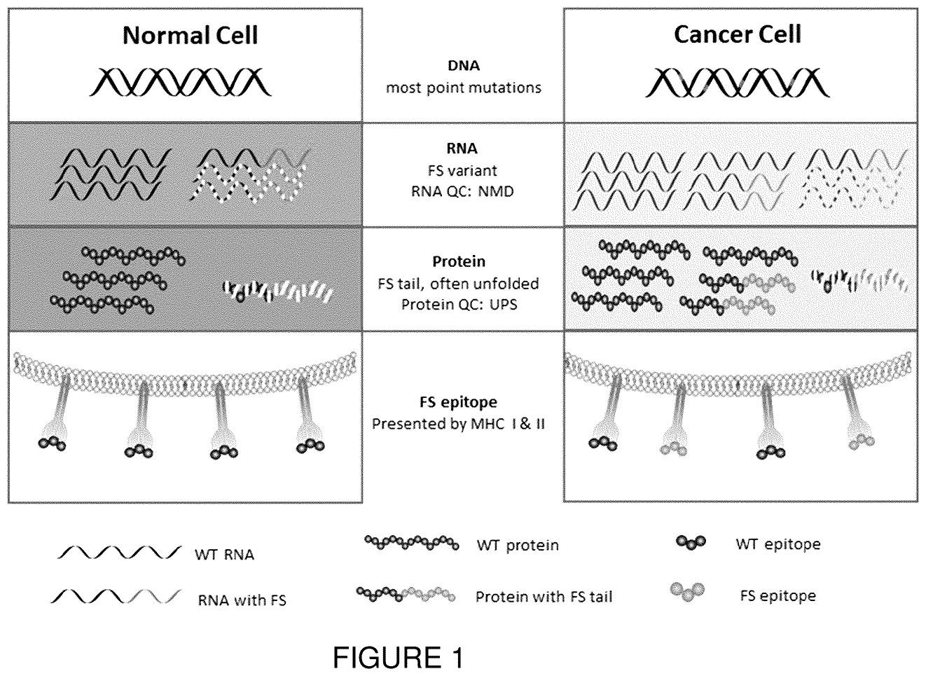

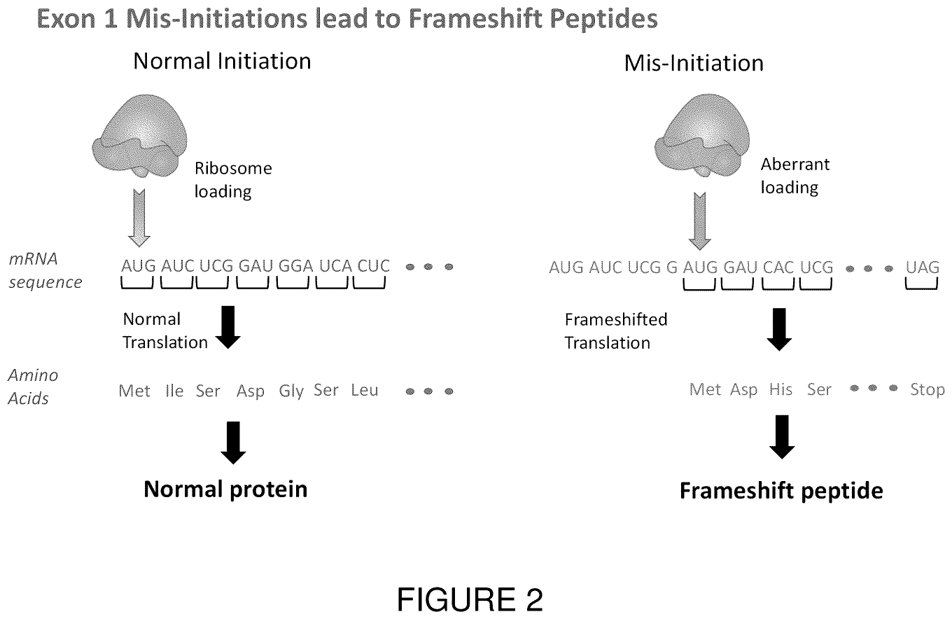

As shown in FIG. 1, FSP formation by mis-initiation at Exon 1 is an error process in cancer cells. In normal cells errors in DNA replication are very rare and repaired. Transcription error rates are higher but also rare as are mis-splicing during intron excision. Additionally, the FS transcript with a premature termination may be degraded by Nonsense Mediated Decay (NMD). Aberrant proteins, including those with frameshifts are largely eliminated by the protein quality control system, Ubiquitin/Proteasome System (UPS). The net result is that in normal cells very few frameshift peptides are presented on MHC I/II or escape the cell to be presented to the immune system. Cancer Cell: All levels of information transfer become more error prone. More errors are made in DNA replication, but only when cells divide. Most DNA mutations are point mutations and encode low or non-immunogenic epitopes. Global transcription is increased and is generally less accurate and even more so through MSs producing INDELs. Most transcribed genes with MSs in the coding region will have more FS transcripts. RNA splicing is also far less accurate, creating more FS transcripts from each out-of-frame splicing between exons from the same gene and different genes. Relevant here, there is a large increase in mis-initiation of translation in tumor cells. Some of these mis-initiations will create FSPs. The substantial increase of the FS transcripts including from exon 1 mis-initiation overwhelms the RNA quality control systems, such as NMD. Consequently, more truncated proteins with the FS peptide will be translated. These unfolded truncated proteins, combined with aberrant proteins from other mutations, overwhelms the protein quality control system, leading to more frameshift peptides being presented on MHC I/II and mis-secreted or released from the cancer cell which the immune system can respond to. FIG. 2 depicts the process by which mis-initiation occurs in cancer cells versus normal cells.

Personal cancer vaccines are promising as a new therapeutic treatment. These vaccines are currently based on mutations in tumor DNA. In some embodiments, variants in RNA production and translation create frameshift neoantigens that may be another source of neoantigens for personal vaccines. Because there are only 34,733 of these antigens possible from mis-initiation at exon 1, a simple peptide array can be used to detect antibodies generated against the exon 1 FSPs.

Checkpoint inhibitor immunotherapeutics are revolutionizing cancer therapy. However, even in the most responsive cancers a substantial portion (50%-80%) of the patients have poor to no positive response. A surprising finding in the analysis of these patients was that one of the best correlates of response has been the total number of neoantigens in the tumor. This is also the case for patients with high microsatellite instability (MSI) where the production of FS neoantigens drives the effective anti-tumor immune responses. The realization of the immunological importance of these DNA mutations has spawned the effort to develop personal vaccines. As promising as early studies are of these vaccines, a major problem is that the majority of tumors will not have enough neoantigen-generating mutations to sustain development of a personal vaccine. For example, melanoma tumors have a high mutational level with an average of 200 neoepitope mutations. This provides a large number to algorithmically screen for optimal antigenic presentation. In recent reports of two Phase I clinical trials of personal melanoma vaccines, starting with 90 to about 2,000 personal neoantigens, 10 or 20 were identified for the vaccine. However, in glioblastoma multiforme (GBM) only 3.5% patients had a high tumor mutation load, and further analysis showed that only a very small subset of GBM patients would potentially benefit from checkpoint blockade treatment. This is also consistent with a lack of response in GBM patients to checkpoint inhibitors. Massive genomic sequencing results indicated that GBM, ovarian cancer, breast adenocarcinoma and many other cancer types had very low number non-synonymous mutations, which will make these cancers difficult targets for personalized cancer vaccines.

To solve this problem, methods and compositions are provided herein related to an alternative source of neoantigens which expand the scope of the application and efficacy of the neoantigen based cancer vaccines. In the process of becoming a tumor, not only does the DNA mutation rate increase with faster cell divisions, but also there is a disruption of basic cellular functions, including RNA transcription, splicing, the correct initiation of translation, and the quality control system on peptides. These processes, combined with the disrupted quality control system in tumor cells, can lead to the production of FS peptides and exposure of the FS epitopes to the immune system. Embodiments provided herein relate to FSPs produced by errors in RNA translation as a source of cancer neoantigens and a simple system to detect them.

Described herein are frameshift peptides resulting from exon 1 mis-initiation of translation (exon 1 FSPs). SEQ ID NOs: 1-19,997 are frameshift peptides resulting from mis-initiation of translation at exon 1. SEQ ID NOs: 1-10,025 are the subset of these that showed reactivity with lung cancer patient samples, as described in Example 3. SEQ ID NOs: 1-32 are the subset that were associated with a patient's response to immunotherapeutics, as described in Example 3. Disclosed herein are peptide arrays which include one or more exon 1 FSPs. Further disclosed herein are vaccines including one or more exon 1 FSPs. Further disclosed herein are therapeutic molecules which bind one or more exon 1 FSPs. In some embodiments, the exon 1 FSPs include one or more peptides having a sequence set forth in SEQ ID NOs: 1-19,997. In some embodiments, the exon 1 FSPs include one or more peptides having a sequence set forth in SEQ ID NOs: 1-10,025. In some embodiments, the exon 1 FSPs include one or more peptides having a sequence set forth in SEQ ID NOs: 1-32.

Disclosed herein are models for how errors in translation initiation could create frameshift neoantigens. Using an array comprising all predicted FS peptides with specific qualifications, human sera from patients with 5 different cancers have higher antibody reactivity than people without cancer. Three different patterns of high antibody reactive can be determined-pan-cancer, cancer-type focused, and personal.

The methods and compositions provided herein indicates that FSPs produced at the RNA level in tumor cells may be a good source of neoantigens for vaccines for several reasons. First, these FSPs produce neoantigens which are more likely to be immunogenic than neo-epitopes encoded by single nucleotide mutations. Second, because of the predictable number of candidates it should be possible to use a peptide array to screen for immune reactive neoantigens. This approach would be much simpler than sequencing tumor DNA obtained from a biopsy. Third, because any expressed gene has the potential to produce neoantigens, it may not be necessary to limit the vaccine to oncological driver genes. Finally, it should be difficult for the tumor cells to evolve away from the vaccine since these FSPs are variants, not heritable mutations. Particularly if the FS antigen was produced in RNA from an essential gene, the tumor cells would need to restrict MHC presentation or create an immune suppressive environment to escape an immune response.

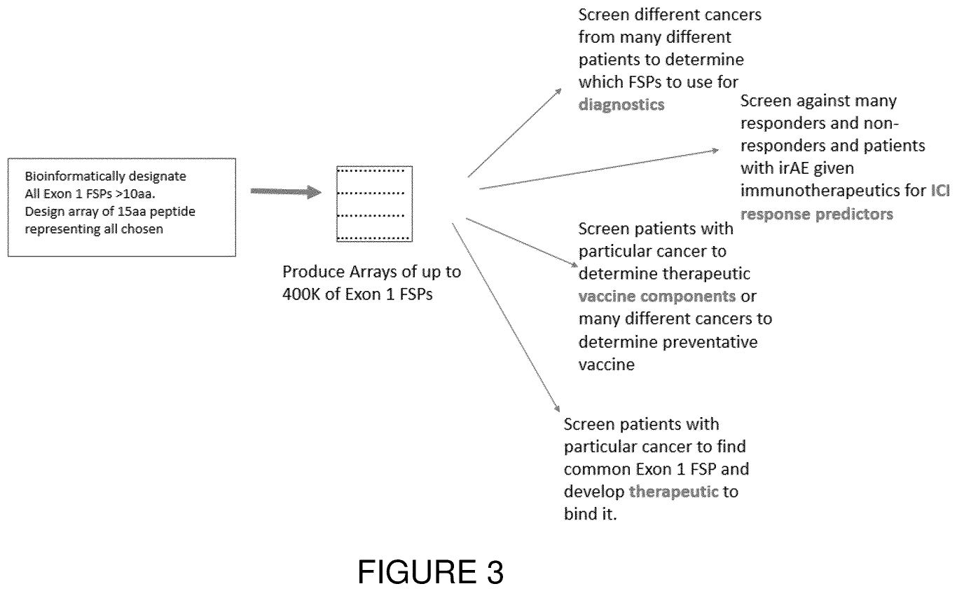

Some embodiments provided herein relate to methods of screening all FSPs that can be formed by mis-initiation of translation, which is then used to develop cancer vaccines, for therapeutics, or for diagnostics. One embodiment of such methods of screen is set forth in FIG. 3, which depicts bioinformatically designating all exon 1 FSPs greater than 10 amino acids in length. An array of 15 amino acid peptides is designed, representing all exon 1 FSPs that were selected. The arrays may include up to 400,000 exon 1 FSPs. For example, the peptide arrays may include about 5000, about 7500, about 10000, about 12500, about 15000, about 17500, about 20000, about 22500, about 25000, about 27500, about 30000, about 32500, about 35000, about 37500, about 40000, about 50000, about 100000, about 200000, about 300000, or about 400000 FSPs. The arrays may then be used in a variety of applications, as shown in FIG. 3, including for diagnostics, immune checkpoint inhibitor (ICI) response predictors, vaccine components, or therapeutics. As a diagnostic, for example, the arrays may be used by screening different cancers from many different patients to determine which FSPs to use in diagnostics. In ICI response predictors, the arrays may be used to screen against many responders and non-responders and patients with an immune related adverse event (irAE) given immunotherapeutics for ICI respond predictors. For vaccines, the arrays may be used by screening patients with a particular cancer to determine therapeutic vaccine components or many different cancers to determine preventative vaccines. For therapeutics, the arrays may be used by screening patients with particular cancers to find common exon 1 FSPs and thereby develop therapeutics to bind the common exon 1 FSPs.

Elements of the model are supported by other published work. The immunological reactivity of FS neoantigens is the presumed basis of the effectiveness of PD-1 in most MSI-H cancers. It also explains the responsiveness of renal cancer to CPI therapy—these cancers have low point mutation levels but high FS mutations.

An important aspect of this source of neoantigens is that it may allow extending the personal vaccines to more patients and tumor types. Many tumors have relatively low numbers of DNA mutations and probably could not support constructing a vaccine. Estimates from published mutational surveys of various tumors indicate that only 40% of patients could be treated with personal vaccines. However, the methods and compositions provided herein predict that the RNA FSPs would be produced in any cancer type, even if the DNA mutation level is low.

The model also predicts that there may be recurrent FSs produced in different tumors. This is substantiated, for example, in the table of FIG. 5, showing that recurrent FSPs from exon 1 mis-initiation can be used to diagnose breast cancer and compose a potential general vaccine for breast cancer.

Sets of FS peptides were found that had enriched activity in individual tumor types. A collection of a set of these peptides could potentially be used to constitute a general, therapeutic vaccine or one focused on a particular tumor or set of tumors. Such vaccines would have an advantage over a personal vaccine of being pre-made but would have fewer antigens in common with the tumor. Conceivably, pan-cancer peptides could be used to create a prophylactic cancer vaccine, as has been proposed for cancer associated antigens. However, as shown in comparing late and early-stage pancreatic cancer profiles, a prophylactic vaccine from FS neoantigens would be best constituted from peptides reactive at early stages of cancer. Clinical trials in dogs were recently initiated of a prophylactic vaccine that is designed to be broadly protective. Accordingly, embodiments provided herein relate to therapeutic or prophylactic vaccines that are developed using the FSPs identified herein.

In summary, the methods and compositions provided herein relate to another class of neoantigens that are useful in developing different types of cancer vaccines. Also provided herein are array formats for directly detecting immune responses to these tumor antigens.

Some embodiments provided herein relate to methods and compositions for classification and characterization of subjects with respect to their likely response to treatment with an immunotherapeutic. In some embodiments, the methods include predicting a subject's response to treatment with an immune checkpoint inhibitor (ICI) therapy. In some embodiments, the methods include contacting a biological sample from the subject to an array as described herein. In some embodiments, the array comprises frameshift peptides (FSPs) resulting from exon 1 mis-initiation of translation.

Some embodiments provided herein relate to methods of measuring antibody affinity to a plurality of peptides. In some embodiments, the methods include contacting a biological sample from the subject to a peptide array comprising one or more exon 1 frameshift peptides. In some embodiments, the subject has cancer. In some embodiments, the biological sample is a blood sample. In some embodiments, the biological sample is a peripheral blood sample.

In some embodiments, the peptide array includes 400,000 total peptides. For example, the peptide arrays may include about 5000, about 7500, about 10000, about 12500, about 15000, about 17500, about 20000, about 22500, about 25000, about 27500, about 30000, about 32500, about 35000, about 37500, about 40000, about 50000, about 100000, about 200000, about 300000, or about 400000 peptides.

In some embodiments, the peptide array may further include other peptides that are not from exon 1 frameshift peptide mis-initiation, such as frameshift peptides caused by RNA processing error such as exon mis-splicing. In some embodiments, the peptide array further includes frameshift peptides caused by indels in transcription.

In some embodiments, the one or more exon 1 frameshift peptides include a peptide having a sequence selected from any one of SEQ ID NO: 1-19,997. In some embodiments, the one or more exon 1 frameshift peptides include a peptide having a sequence selected from any one of SEQ ID NO: 1-10,025. In some embodiments, the one or more exon 1 frameshift peptides include a peptide having a sequence selected from any one of SEQ ID NO: 1-32. In some embodiments, the one or more exon 1 frameshift peptides include a peptide having at least 90% sequence identity to any one of SEQ ID NO: 1-19,997. In some embodiments, the one or more exon 1 frameshift peptides include a peptide having at least 95% sequence identity to any one of SEQ ID NO: 1-19,997. In some embodiments, the one or more exon 1 frameshift peptides include a peptide having at least 99% sequence identity to any one of SEQ ID NO: 1-19,997. In some embodiments, the one or more exon 1 frameshift peptides include a peptide having at least 90% sequence identity to any one of SEQ ID NO: 1-10,025. In some embodiments, the one or more exon 1 frameshift peptides include a peptide having at least 95% sequence identity to any one of SEQ ID NO: 1-10,025. In some embodiments, the one or more exon 1 frameshift peptides include a peptide having at least 99% sequence identity to any one of SEQ ID NO: 1-10,025. In some embodiments, the one or more exon 1 frameshift peptides include a peptide having at least 90% sequence identity to any one of SEQ ID NO: 1-32. In some embodiments, the one or more exon 1 frameshift peptides include a peptide having at least 95% sequence identity to any one of SEQ ID NO: 1-32. In some embodiments, the one or more exon 1 frameshift peptides include a peptide having at least 99% sequence identity to any one of SEQ ID NO: 1-32.

In some embodiments, the methods further include measuring the presence or absence of antibodies having affinity to one or more of the FSPs resulting from exon 1 mis-initiation of translation. In some embodiments, measuring comprises measuring binding of antibodies in the biological sample, wherein the antibodies have affinity to the one or more exon 1 frameshift peptides. In some embodiments, measuring is performed with a device, such as a laser scanner or other fluorescence imager. In some embodiments, measuring is qualitative, such as, for example, the presence or absence of antibodies is measured. In some embodiments, measuring is quantitative, for example, intensity or strength of binding is measured.

In some embodiments, the methods further include quantifying a level or levels of the antibodies having affinity to one or more of the exon 1 frameshift peptides. In some embodiments, quantifying is performed electrically, for example, with a device that receives measurements of binding and outputs a numerical value (level) corresponding to the measured binding. In some embodiments, levels of binding are quantified for each of one or more exon 1 frameshift peptides.

In some embodiments, the methods further include generating an exon 1 signature of the subject based on the quantified level or levels of binding to the plurality of the exon 1 peptides. In some embodiments, the exon 1 signature is computationally generated. In some embodiments, the exon 1 signature is predictive of a clinical outcome of an immunotherapeutic treatment in the subject.

Some embodiments provided herein relate to methods of classifying whether a subject might experience an immune-related adverse event in response to treatment. In some embodiments, the methods provided herein include the steps of: (a) contacting a biological sample from the subject to a frameshift array comprising a plurality of exon 1 frameshift peptides; (b) detecting the presence or absence of antibodies having affinity to one or more of the exon 1 frameshift peptides in the contacted biological sample; (c) quantifying a level of the antibodies having affinity to one or more of the exon 1 frameshift peptides to form an exon 1 signature of the subject; and (d) classifying the subject as having a high likelihood of experiencing an adverse event based on comparison of the subject's exon 1 binding pattern to one or more standards, wherein the signature distinguishes a person likely to have an adverse event from those that are unlikely to have an adverse event in response to immunotherapy.

In some embodiments, the immunotherapeutic is a cytotoxic T-lymphocyte-associated protein 4 (CTLA-4) inhibitor, a programmed death-ligand 1 (PD-L1) inhibitor, a programmed cell death protein 1 (PD-1) inhibitor, an OX40 agonist, an antibody to B7 ligands, or a BY55 monoclonal antibody. In some embodiments, the subject has already received treatment with the immunotherapeutic. In some embodiments, the subject has not yet received treatment with an immunotherapeutic.

In some embodiments, the methods further include creating a record indicating the subject is likely to respond to the immunotherapeutic treatment based on the exon 1 signature. In some embodiments, the methods further include creating a record indicating the subject is not likely to respond to the immunotherapeutic treatment based on the exon 1 signature. In some embodiments, the methods further include creating a record indicating the subject is likely to have an adverse event to the immunotherapeutic treatment based on the exon 1 signature. In some embodiments, the methods further include creating a record indicating the subject is not likely to have an adverse event to the immunotherapeutic treatment based on the exon 1 signature. In some embodiments, the record is created on a computer readable medium.

As used herein, the term “substrate” refers to any type of solid support to which the peptides are immobilized. Examples of substrates include, but are not limited to, microarrays; beads; columns; optical fibers; wipes; nitrocellulose; nylon; glass; quartz; diazotized membranes (paper or nylon); silicones; polyformaldehyde; cellulose; cellulose acetate; paper; ceramics; metals; metalloids; semiconductive materials; coated beads; magnetic particles; plastics such as polyethylene, polypropylene, and polystyrene; gel-forming materials; silicates; agarose; polyacrylamides; methylmethracrylate polymers; sol gels; porous polymer hydrogels; nanostructured surfaces; nanotubes (such as carbon nanotubes); and nanoparticles (such as gold nanoparticles or quantum dots). When bound to a substrate, the peptides can be directly linked to the support, or attached to the surface via a linker. Thus, the solid substrate and/or the peptides can be derivatized using methods known in the art to facilitate binding of the peptides to the solid support, so long as the derivatization does not eliminate detection of binding between the peptides and antibodies in the sera.

As used herein, the term “sample” means non-biological samples and biological samples. Non-biological samples include those prepared in vitro comprising varying concentrations of a target molecule of interest in solution. Biological samples include, without limitation, blood, lymph, urine, saliva, sputum, other bodily secretions, cells, and tissue specimens and dilutions of them. Any suitable biological sample can be used. For example, a biological sample can be a specimen obtained from a subject (for example, a mammal such as a human, canine, mouse, rat, pig, guinea pig, cow, monkey, or ape) or can be derived from such a subject. A subject can provide a plurality of biological samples. A biological sample also can be a biological fluid such as urine, blood, plasma, serum, saliva, tears, or mucus, or such a sample absorbed onto a paper or polymer substrate. A biological sample can be further fractionated, if desired, to a fraction containing particular cell types. In some cases, sera are obtained from the individual using techniques known in the art. The biological sample can be a blood, tissue, or other bodily sample. The blood sample can be a peripheral blood sample.

Embodiments of the methods provided herein may be sensitive and involve small quantities of biological samples from a subject. In some embodiments, biological samples from a subject are too concentrated and require a dilution prior to being contacted with an array of the present disclosure. A plurality of dilutions can be applied to a biological sample prior to contacting the sample with an array of the present disclosure. A dilution can be a serial dilution, which can result in a geometric progression of the concentration in a logarithmic fashion. For example, a ten-fold serial dilution can be 1 M, 0.01 M, 0.001 M, and a geometric progression thereof. A dilution can be, for example, a one-fold dilution, a two-fold dilution, a three-fold dilution, a four-fold dilution, a five-fold dilution, a six-fold dilution, a seven-fold dilution, an eight-fold dilution, a nine-fold dilution, a ten-fold dilution, a sixteen-fold dilution, a twenty-five-fold dilution, a thirty-two-fold dilution, a sixty-four-fold dilution, and/or a one-hundred-and-twenty-five-fold dilution, or any dilution appropriate for the required analysis.

The peptide array can be contacted with the biological sample under any suitable conditions to promote binding of antibodies in the sample to peptides immobilized on the array. Thus, the methods presented herein are not limited by any specific type of binding conditions employed. Such conditions will vary depending on the array being used, the type of substrate, the density of the peptides arrayed on the substrate, desired stringency of the binding interaction, and nature of the competing materials in the binding solution. In a certain embodiments, the conditions comprise a step to remove unbound antibodies from the addressable array.

Similarly, any suitable detection technique can be used in the methods provided herein to detect binding of antibodies in the biological sample to peptides on the array to generate a subject's exon 1 signature. In one embodiment, any type of detectable label can be used to label peptides on the array, including but not limited to radioisotope labels, fluorescent labels, luminescent labels, and electrochemical labels (such as, for example, ligand labels with different electrode mid-point potential, where detection comprises detecting electric potential of the label). Alternatively, bound antibodies can be detected, for example, using a detectably labeled secondary antibody.

As used herein, the term “detect,” “detection,” “detectable,” or “detecting” has its ordinary meaning as understood in light of the specification, and is understood both on a quantitative and a qualitative level, as well as a combination thereof. It thus includes quantitative, semi-quantitative, and qualitative measurements of measuring a cancer in a subject, using the methods and compositions as disclosed herein.

As used herein, the expression “a subject” has its ordinary meaning as understood in light of the specification, and means a human or non-human mammal. The subject may exhibit one or more symptoms or indications of cancer, and/or have been diagnosed with cancer, including a solid tumor and may need treatment for the same. For example, the subject may be a human or canine. In many embodiments, the term “subject” may be interchangeably used with the term “patient.” For example, a human subject may be diagnosed with a primary or a metastatic tumor and/or with one or more symptoms or indications including, but not limited to, unexplained weight loss, general weakness, persistent fatigue, loss of appetite, fever, night sweats, bone pain, shortness of breath, swollen abdomen, chest pain/pressure, enlargement of spleen, and elevation in the level of a cancer-related biomarker.

The term “malignancy” has its ordinary meaning as understood in light of the specification, and refers to a non-benign tumor or a cancer. As used herein, the term “cancer” includes a malignancy characterized by deregulated or uncontrolled cell growth. Exemplary cancers include carcinomas, sarcomas, leukemias, and lymphomas. Cancer includes primary malignant tumors (for example, those whose cells have not migrated to sites in the subject's body other than the site of the original tumor) and secondary malignant tumors (for example, those arising from metastasis, the migration of tumor cells to secondary sites that are different from the site of the original tumor). A cancer may include, for example, gastric, myeloid, colon, nasopharyngeal, esophageal, and prostate tumors, glioma, neuroblastoma, breast cancer, lung cancer, ovarian cancer, colorectal cancer, thyroid cancer, leukemia (for example, Adult T-cell leukemia, Acute monocytic leukemia, Acute myeloid leukemia, Acute promyelocytic leukemia, myelogenous leukemia, lymphocytic leukemia, acute myelogenous leukemia (AML), chronic myeloid leukemia (CML), acute lymphoblastic leukemia (ALL), T-lineage acute lymphoblastic leukemia or T-ALL chronic lymphocytic leukemia (CLL), myelodysplastic syndrome (MDS), hairy cell leukemia), lymphoma (Hodgkin's lymphoma (HL), non-Hodgkin's lymphoma (NHL)), multiple myeloma, bladder, renal, gastric (for example, gastrointestinal stromal tumors (GIST)), liver, melanoma and pancreatic cancer, sarcoma, Adenocarcinoma, Astrocytoma, Bone Cancer, Brain Tumor, Burkitt's lymphoma, Carcinoma, Cervical Cancer, Chronic Lymphocytic Leukemia, Chronic myelogenous leukemia, Endometrial cancer, Glioblastoma multiforme, Glioma, Hepatocellular carcinoma, Hodgkin's lymphoma, Inflammatory breast cancer, Kidney Cancer, Leukemia, Lymphoma, Malignant Mesothelioma, Medulloblastoma, Melanoma, Multiple myeloma, Neuroblastoma, Non-Hodgkin Lymphoma, Non-Small Cell Lung Cancer, Pancreatic Cancer, Pituitary tumor, Retinoblastoma, Skin Cancer, Small Cell Lung Cancer, Squamous cell carcinoma, Stomach cancer, T-cell leukemia, T-cell lymphoma, and Wilms' tumor.

As used herein the term “frameshift mutation” has its ordinary meaning as understood in light of the specification, and is a mutation or variant causing a change in the reading frame of the RNA. Thus, a FSP is a peptide in which a frame has changed due to a frameshift mutation or variant. In some embodiments provided herein, a frameshift includes two or more pooled frameshifts. As used herein, the term “pooled” refers to a plurality of frameshift samples that have been combined to create a new composition. In some cases, the plurality of FSPs comprise one or more peptides as set forth in SEQ ID NOs: 1-19,997, which are the potential FSPs predicted from exon 1 mis-initiation as based on current annotated sequences in humans. In some cases, the plurality of FSPs comprise one or more peptides as set forth in SEQ ID NOs: 1-10,025. In some cases, the plurality of FSPs comprise one or more peptides as set forth in SEQ ID NOs: 1-32,

As used herein the term “immunotherapeutic” or “IT” has its ordinary meaning as understood in light of the specification, and refers to a compound such as a therapeutic molecule that is used to, in this case, treat cancer by inducing, enhancing or suppressing the immune response. Immunotherapeutics encompass immune checkpoint inhibitors, antibody-drug conjugates (ADCs), monoclonal antibodies, T-cell therapy, small molecules, and bispecific antibodies (bsAbs). Antibody-drug conjugates include monoclonal antibodies linked to biologically active drugs to combine the targeting ability of antibodies as well as the cytotoxic ability of the drug. T-cell therapy involves reprogramming a patient's own immune T cells to attack tumors. One type of well-known T-cell therapy comprises adoptive transfer of chimeric antigen receptor (CAR) T-cells. As used herein, the term “chimeric antigen receptor” has its ordinary meaning as understood in light of the specification, and refers to a fusion protein of the membrane or intracellular signaling region of T-cell activating proteins (for example, CD3-zeta chain, CD28, 41BBL, OX40, ICOS, high-affinity receptor for IgE (FcεcRI) and other T-cell activating proteins) and the antigen-binding site (such as, for example, single-chain Fv fragment) of a cancer antigen-specific antibody. Bispecific antibodies are recombinant proteins that can bind to two different types of antigen at the same time. For example, a bsAb can be engineered to bind a cytotoxic cell and a target tumor cell. That way, the bsAb brings the cytotoxic cell and the target tumor cell into close proximity and facilitates tumor treatment.

In certain embodiments, the immunotherapeutic is selected from Tremelimumab (CTLA-4 blocking antibody), OX40 agonists (for example, agonist antibodies), antibodies to B7 ligands (for example, anti-B7-H1, anti-B7-H3, anti-B7-H3, anti-B7-H4), durvalumab (MEDI4736, anti-PD-L1 antibody), MK-3475 (PD-1 blocker), Nivolumab (anti-PD-1 antibody), Pembrolizumab (anti-PD-1 antibody), Pidilizumab/CT-011, BY55 monoclonal antibody, AMP224 (anti-PD-L1 antibody), BMS-936559 (anti-PD-L1 antibody), MPLDL3280A (anti-PD-L1 antibody), MSB0010718C (anti-PD-L1 antibody), and Yervoy/ipilimumab (anti-CTLA-4 checkpoint inhibitor). Many new inhibitor targets are being investigated. In some cases, IT treatment comprises a combination therapy in which two or more immunotherapeutics are administered.

As used herein the terms “checkpoint inhibitor” and “checkpoint pathway inhibitor” are used interchangeably and has its ordinary meaning as understood in light of the specification, and refer to negative regulatory molecules, usually antibodies, that block or inhibit anti-T cell anti-tumor function to enhance tumor killing. Checkpoint inhibitors include, without limitation, CTLA-4, PD-L1, PD-L2, PD-1, B7-H3, B7-H4, BTLA, HVEM, TIM3, GAL9, LAG3, VISTA, KIR, 2B4, CD160, CGEN-15049, CHK1, CHK2, A2aR, and a B-7 family ligand such as B7-1, B7-2, B7-DC, B7-H1, B7-H2, B7-H3, B7-H4, B7-H5, B7-H6 and B7-H7 (or any combination thereof), or a combination thereof (for example, a combination of CTLA-4 and PD-L1 or PD-L2).

As used herein, the term “side effect,” “side effects,” “adverse event,” or “adverse events” has its ordinary meaning as understood in light of the specification, and refers to the unacceptable or undesirable adverse symptoms resulting from or associated with the administration of a particular treatment such as an IT therapy. Side effects specifically to immunotherapeutics are termed “immune related adverse events” (irAE). While side effects vary by the type of therapy, common side effects of IT therapies include, without limitation fatigue, infusion related reactions, dermatological toxicity, diarrhea/colitis, hepatotoxicity, pneumonitis, hyper- and hypo-thyroidism. Immune-related adverse events are generally graded from 1-4. Grades 3 and 4 are considered serious and can require immunosuppression treatment. Patients with irAE are just as likely to have a positive response to treatment. Occurrence of Grade 3 or 4 event can prohibit the patient from further IT therapy. Therefore, knowing ahead of time which patients are more likely to have an event would allow closer monitoring to preempt a Grade 3 or 4 event.

Any appropriate criteria can be used to confirm a subject's responsiveness to treatment with an IT. For example, in certain embodiments, responsiveness to treatment by an IT is measured by at least one criterion selected from the group consisting of clinical benefit rate, survival until mortality, pathological complete response, semi-quantitative measures of pathologic response, clinical complete remission, clinical partial remission, clinical stable disease, recurrence-free survival, metastasis free survival, disease free survival, circulating tumor cell decrease, circulating marker response, and RECIST criteria.

Some embodiments provided herein relate to methods of treating or preventing cancer. In some embodiments, the methods include administering a therapeutic molecule. In some embodiments, the therapeutic molecule is designed to bind frameshift peptides (FSPs) resulting from exon 1 mis-initiation of translation. In some embodiments, the therapeutic molecule is an antibody or synthetic antibody. In some embodiments, the therapeutic molecule includes a fragment of an antibody (such as a variable region (“scFv”)) or a derivative of an antibody or a fragment of an antibody, such as a CAR-T cell.

In some embodiments, the therapeutic molecule binds a FSP resulting from exon 1 mis-initiation of translation. In some embodiments, the FSP includes one or more peptides as set forth in SEQ ID NOs: 1-19,997. In some embodiments, the FSP includes one or more peptides having at least 90% sequence identity to any one of SEQ ID NO: 1-19,997. In some embodiments, the FSP includes one or more peptides having at least 95% sequence identity to any one of SEQ ID NO: 1-19,997. In some embodiments, the FSP includes one or more peptides having at least 99% sequence identity to any one of SEQ ID NO: 1-19,997. In some embodiments, the FSP includes one or more peptides as set forth in SEQ ID NOs: 1-10,025. In some embodiments, the FSP includes one or more peptides having at least 90% sequence identity to any one of SEQ ID NO: 1-10,025. In some embodiments, the FSP includes one or more peptides having at least 95% sequence identity to any one of SEQ ID NO: 1-10,025. In some embodiments, the FSP includes one or more peptides having at least 99% sequence identity to any one of SEQ ID NO: 1-10,025. In some embodiments, the FSP includes one or more peptides as set forth in SEQ ID NOs: 1-32. In some embodiments, the FSP includes one or more peptides having at least 90% sequence identity to any one of SEQ ID NO: 1-32. In some embodiments, the FSP includes one or more peptides having at least 95% sequence identity to any one of SEQ ID NO: 1-32. In some embodiments, the FSP includes one or more peptides having at least 99% sequence identity to any one of SEQ ID NO: 1-32.

In some embodiments, the therapeutic molecule is a vaccine. Some embodiments relate to administering a vaccine. In some embodiments, the vaccine comprises FSPs resulting from exon 1 mis-initiation of translation. In some embodiments, the FSPs comprise one or more peptides as set forth in SEQ ID NOs: 1-19,997. In some embodiments, the FSPs include one or more peptides having at least 90% sequence identity to any one of SEQ ID NO: 1-19,997. In some embodiments, the FSPs include one or more peptides having at least 95% sequence identity to any one of SEQ ID NO: 1-19,997. In some embodiments, the FSPs include one or more peptides having at least 99% sequence identity to any one of SEQ ID NO: 1-19,997. In some embodiments, the FSPs comprise one or more peptides as set forth in SEQ ID NOs: 1-10,025. In some embodiments, the FSPs include one or more peptides having at least 90% sequence identity to any one of SEQ ID NO: 1-10,025. In some embodiments, the FSPs include one or more peptides having at least 95% sequence identity to any one of SEQ ID NO: 1-10,025. In some embodiments, the FSPs include one or more peptides having at least 99% sequence identity to any one of SEQ ID NO: 1-10,025. In some embodiments, the FSPs comprise one or more peptides as set forth in SEQ ID NOs: 1-32. In some embodiments, the FSPs include one or more peptides having at least 90% sequence identity to any one of SEQ ID NO: 1-32. In some embodiments, the FSPs include one or more peptides having at least 95% sequence identity to any one of SEQ ID NO: 1-32. In some embodiments, the FSPs include one or more peptides having at least 99% sequence identity to any one of SEQ ID NO: 1-32.

In some embodiments, biological samples, such as blood, from cancer patients are applied to the FSP arrays described herein to determine reactivity of peptides for each patient. In some embodiments, FSPs unique to the patient are used in a personal vaccine. In some embodiments, FSPs shared between different patients are used for off-the-shelf therapeutic or preventative vaccines. In some embodiments, the vaccine compositions include one or more frameshift peptides (FSPs) resulting from exon 1 mis-initiation of translation. In some embodiments, the FSPs include one or more peptides as set forth in SEQ ID NOs: 1-19,997. In some embodiments, the FSPs include one or more peptides having at least 90% sequence identity to any one of SEQ ID NO: 1-19,997. In some embodiments, the FSPs include one or more peptides having at least 95% sequence identity to any one of SEQ ID NO: 1-19,997. In some embodiments, the FSPs include one or more peptides having at least 99% sequence identity to any one of SEQ ID NO: 1-19,997. In some embodiments, the FSPs include one or more peptides as set forth in SEQ ID NOs: 1-10,025. In some embodiments, the FSPs include one or more peptides having at least 90% sequence identity to any one of SEQ ID NO: 1-10,025. In some embodiments, the FSPs include one or more peptides having at least 95% sequence identity to any one of SEQ ID NO: 1-10,025. In some embodiments, the FSPs include one or more peptides having at least 99% sequence identity to any one of SEQ ID NO: 1-10,025. In some embodiments, the FSPs include one or more peptides as set forth in SEQ ID NOs: 1-32. In some embodiments, the FSPs include one or more peptides having at least 90% sequence identity to any one of SEQ ID NO: 1-32. In some embodiments, the FSPs include one or more peptides having at least 95% sequence identity to any one of SEQ ID NO: 1-32. In some embodiments, the FSPs include one or more peptides having at least 99% sequence identity to any one of SEQ ID NO: 1-32.

In some embodiments, the vaccine compositions further include an adjuvant. In some embodiments, the adjuvant is ABM2, AS01B, AS02, AS02A, Adjumer, Adjuvax, Algammulin, Alum, Aluminum phosphate, Aluminum potassium sulfate, Bordetella pertussis, Calcitriol, Chitosan, Cholera toxin, CpG, Dibutyl phthalate, Dimethyldioctadecylammonium bromide (DDA), Freund's adjuvant, Freund's complete, Freund's incomplete (IFA), GM-CSF, GMDP, Gamma Inulin, Glycerol, HBSS (Hank's Balanced Salt Solution), polyinosinic-polycytidylic acid stabilized with polylysine and carboxymethylcellulose (poly-ICLC, also known as Hiltonol), IL-12, IL-2, Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non-ionic copolymer), Pam3Cys (lipoprotein), Pertussis toxin, Poloxamer, QS21, RaLPS, Ribi, Saponin, Seppic ISA 720, Soybean Oil, Squalene, Syntex Adjuvant Formulation (SAF), Synthetic polynucleotides (poly IC/poly AU), TiterMax Tomatine, Vaxfectin, XtendIII, or Zymosan.

Accordingly, some embodiments are provided herein as set forth in the following numbered alternatives:
1. An array comprising frameshift peptides (FSPs) resulting from exon 1 mis-initiation of translation.
2. The array of alternative 1, wherein the FSPs on the array are spaced between 3 and 9 μm.
3. The array of any one of alternatives 1-2, wherein the array is used to diagnose cancer.
4. The array of any one of alternatives 1-2, wherein the array is used to predict response to immunotherapy.
5. The array of any one of alternatives 1-2, wherein the array is used to predict adverse responses to immunotherapy.
6. The array of any one of alternatives 1-5, wherein the FSPs comprise one or more peptides as set forth in SEQ ID NOs: 1-19,997.
7. The array of any one of alternatives 1-6, wherein the array comprises at least about 2500, about 5000, about 7500, about 10000, about 12500, about 15000, about 17500, about 20000, about 22500, about 25000, about 27500, about 30000, about 32500, about 35000, about 37500, about 40000, about 50000, about 100000, about 200000, about 300000, or about 400000 FSPs.
8. A vaccine composition comprising frameshift peptides (FSPs) resulting from exon 1 mis-initiation of translation.
9. The vaccine composition of alternative 8, further comprising an adjuvant.
10. The vaccine composition of alternative 9, wherein the adjuvant is ABM2, AS01B, AS02, AS02A, Adjumer, Adjuvax, Algammulin, Alum, Aluminum phosphate, Aluminum potassium sulfate, Bordetella pertussis, Calcitriol, Chitosan, Cholera toxin, CpG, Dibutyl phthalate, Dimethyldioctadecylammonium bromide (DDA), Freund's adjuvant, Freund's complete, Freund's incomplete (IFA), GM-CSF, GMDP, Gamma Inulin, Glycerol, HBSS (Hank's Balanced Salt Solution), Hiltonol, IL-12, IL-2, Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non-ionic copolymer), Pam3Cys (lipoprotein), Pertussis toxin, Poloxamer, QS21, RaLPS, Ribi, Saponin, Seppic ISA 720, Soybean Oil, Squalene, Syntex Adjuvant Formulation (SAF), Synthetic polynucleotides (poly IC/poly AU), TiterMax Tomatine, Vaxfectin, XtendIII, or Zymosan.
11. A method of detecting cancer in a subject, the method comprising: contacting a biological sample from the subject to an array comprising frameshift peptides (FSPs) resulting from exon 1 mis-initiation of translation; and measuring antibody reactivity to the FSPs resulting from mis-initiation of translation in tumors.
12. The method of alternative 11, wherein the FSPs comprise one or more peptides as set forth in SEQ ID NOs: 1-19,997.
13. The method of any one of alternatives 11-12, wherein the FSPs are fixed on a substrate.
14. The method of alternative 13, wherein the substrate comprises glass, silica, composite, resin, or combination thereof.
15. The method of any one of alternatives 11-14, wherein the array is configured to detect binding by at least one of fluorescence, luminescence, calorimetry, chromatography, radioactivity, Bio-Layer Interferometry, and surface plasmon resonance.
16. The method of any one of alternatives 11-15, wherein the array comprises at least about 2500, about 5000, about 7500, about 10000, about 12500, about 15000, about 17500, about 20000, about 22500, about 25000, about 27500, about 30000, about 32500, about 35000, about 37500, about 40000, about 50000, about 100000, about 200000, about 300000, or about 400000 FSPs.
17. The method of any one of alternatives 11-16, wherein the biological sample comprises blood, serum, plasma, cerebrospinal fluid, saliva, urine, or combinations thereof.
18. The method of any one of alternatives 11-17, wherein the biological sample comprises an antibody.
19. The method of any one of alternatives 11-18, wherein the subject is a mammal.
20. The method of any one of alternatives 11-19, wherein the subject is a human, a dog, a cat, a mouse, a rat, a rabbit, a horse, a cow, or a pig.
21. The method of any one of alternatives 11-20, wherein the subject is suspected of having a cancer.
22. The method of any one of alternatives 11-21, wherein the cancer is selected from the group consisting of Acute lymphoblastic leukemia, Acute monocytic leukemia, Acute myeloid leukemia, Acute promyelocytic leukemia, Adenocarcinoma, Adult T-cell leukemia, Astrocytoma, Bladder cancer, Bone Cancer, Brain Tumor, Breast Cancer, Burkitt's lymphoma, Carcinoma, Cervical Cancer, Chronic Lymphocytic Leukemia, Chronic myelogenous leukemia, Colon Cancer, Colorectal cancer, Endometrial cancer, Glioblastoma multiforme, Glioma, Hepatocellular carcinoma, Hodgkin's lymphoma, Inflammatory breast cancer, Kidney Cancer, Leukemia, Lung cancer, Lymphoma, Malignant Mesothelioma, Medulloblastoma, Melanoma, Multiple myeloma, Neuroblastoma, Non-Hodgkin Lymphoma, Non-Small Cell Lung Cancer, Ovarian Cancer, Pancreatic Cancer, Pituitary tumor, Prostate cancer, Retinoblastoma, Skin Cancer, Small Cell Lung Cancer, Squamous cell carcinoma, Stomach cancer, T-cell leukemia, T-cell lymphoma, Thyroid cancer, and Wilms' tumor.
23. A method of treating or preventing cancer, comprising administering a therapeutic molecule designed to bind frameshift peptides (FSPs) resulting from exon 1 mis-initiation of translation.
24. The method of alternative 23, wherein the therapeutic molecule is an antibody or synthetic antibody.
25. The method of any one of alternatives 23-24, wherein the therapeutic molecule binds a FSP resulting from exon 1 mis-initiation of translation.
26. The method of any one of alternatives 23-25, wherein the therapeutic molecule is a vaccine.
27. The method of alternative 26, wherein the vaccine comprises FSPs resulting from exon 1 mis-initiation of translation.
28. The method of any one of alternatives 23-27, wherein the FSPs comprise one or more peptides as set forth in SEQ ID NOs: 1-19,997.
29. A method of predicting response to an immunotherapy, comprising identifying frameshift peptides (FSPs) resulting from exon 1 mis-initiation of translation.
30. The method of alternative 29, wherein the method is used to predict adverse immune responses to immunotherapy.
31. The method of any one of alternatives 29-30, wherein the FSPs comprise one or more peptides as set forth in SEQ ID NOs: 1-19,997.

Additional variations than those described herein will be apparent from this disclosure. For example, depending on the embodiment, certain acts, events, or functions of any of the compositions, systems, or methods described herein can be performed in a different sequence, can be added, merged, or left out altogether. Moreover, in certain embodiments, acts or events can be performed concurrently. In addition, different tasks or processes can be performed by different machines and/or computing systems that can function together.

The foregoing description and examples have been set forth merely to illustrate the disclosure and are not intended as being limiting. Each of the disclosed aspects and embodiments of the present disclosure may be considered individually or in combination with other aspects, embodiments, and variations of the disclosure. In addition, unless otherwise specified, none of the steps of the methods of the present disclosure are confined to any particular order of performance. Modifications of the disclosed embodiments incorporating the spirit and substance of the disclosure may occur to persons skilled in the art and such modifications are within the scope of the present disclosure.

### Examples

Embodiments described herein are further defined in the following Examples. It should be understood that these Examples are given by way of illustration only. From the above discussion and these Examples, one skilled in the art can ascertain the essential characteristics of this disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims. The disclosure of each reference set forth herein is incorporated herein by reference in its entirety.

### Example 1: FSP Analysis

To identify potential putative transcripts, that when translated would result in a frame-shifted neo-peptide, an algorithm was applied to two publicly available datasets to identify FSPs that would result from mis-initiation of translation at the exon 1 of each gene. FSP predicted to be less than 8 amino acids long or were homologous to normal protein sequences were eliminated.

A total number of 64 non-cancer control samples and 13 pancreatic stage 1 cancer samples, 85 late-stage cancer samples from 5 cancer types were tested on the FS array, detailed information was summarized in Table 1. All samples were acquired from collaborators and were informed consent upon collection through the institute's own Institutional Review Board (IRB). All samples were anonymized before receipt at Arizona State University (ASU) via IRB protocol No. STUDY00003722, ‘Receipt of Deidentified Human Serum for Immunosignature Analysis’ and protocol No. 0912004625, ‘Profiling Biological Sera for Unique Antibody Signatures’. All experiments were performed in accordance with the approval protocol.

TABLE 1
Samples tested on Human 400K FS array

| | Number | |
|---|---|---|
| Sample Type | of Samples | Source |
| Breast Cancer (BC) | 17 | UT Southwestern |
| Lung Cancer (LC) | 17 | UT Southwestern |
| GBM | 17 | Barrows Neurological Institute |
| Pancreatic Cancer (PC) | 17 | TGEN |
| Pancreatic Cancer Stage 1 | 13 | TGEN |
| Gastric Cancer (GC) | 17 | Japan |
| Control | 64 | Varied Sources |

### 400K Frameshift Peptide Array Assay

Serum was diluted 1:100 in binding buffer (0.01M Tris-HCl, pH 7.4, 1% alkali-soluble casein, 0.05% Tween-20) and 150 μl diluted samples were loaded into each compartment of the 12-plex array and incubated overnight at room temperature or 4° C. After sample binding, the arrays were washed 3× in wash buffer (1× TBS, 0.05% Tween-20), 10 min per wash. Primary sample binding was detected via Alexa Fluor® 647-conjugated goat anti-human IgG secondary antibody (Jackson ImmunoResearch #109-605-098). The secondary antibody was diluted 1:10,000 (final concentration 0.15 ng/μl) in secondary binding buffer (1× TBS, 1% alkali-soluble casein, 0.05% Tween-20). Arrays were incubated with secondary antibody for 3 h at room temperature, washed 3× in wash buffer (10 min per wash), 30 secs in reagent-grade water, and then dried by centrifuging at 690 RPM for 5 mins. All washes and centrifugations were done on a Little Dipper 650C Microarray Processor (SciGene) with preset programs. Fluorescent signal of the secondary antibody was detected by scanning at 635 nm at 2 μm resolution and 15% gain, using an MS200 microarray scanner (Roche NimbleGen). The 34,733 predicted possible exon 1 FSPs were included in the 400K peptide array. The remainder of the FSPs were predicted from exon mis-splicing or INDELs in encoded microsatellites.

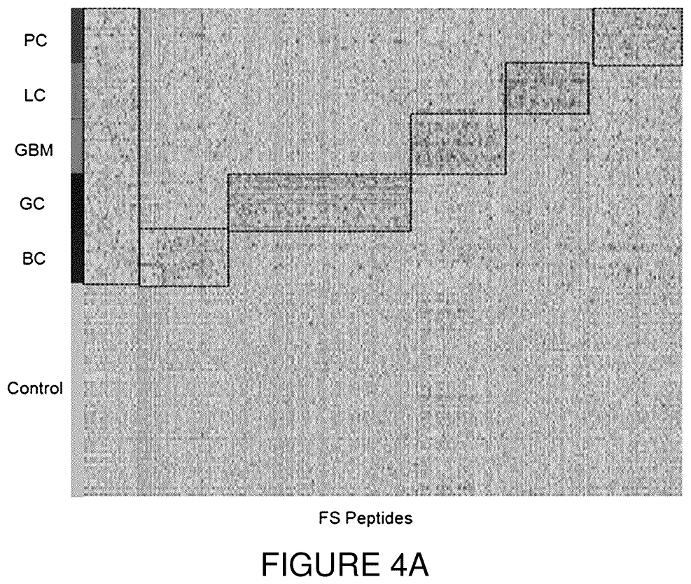

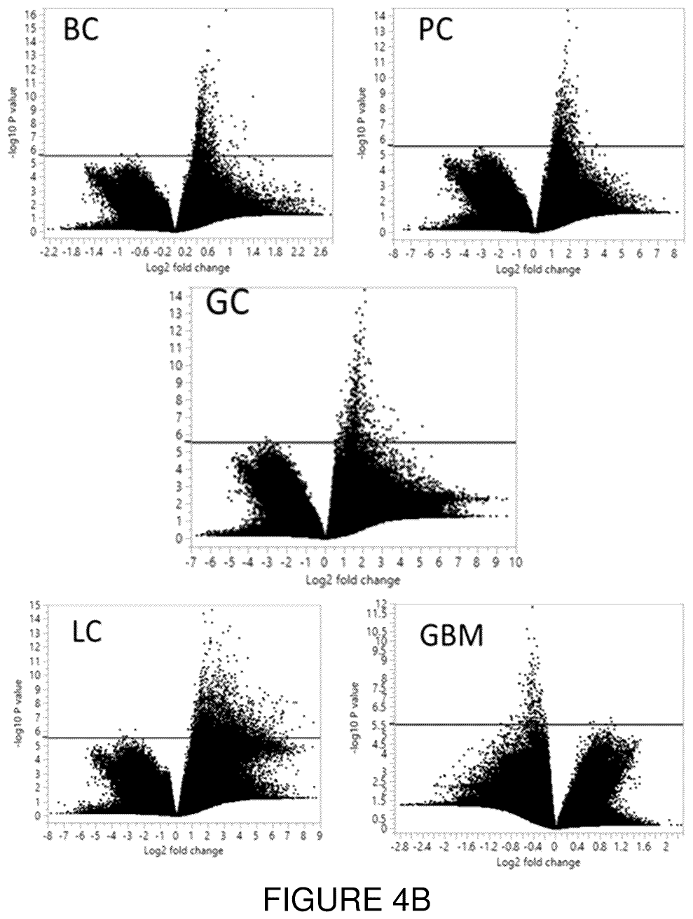

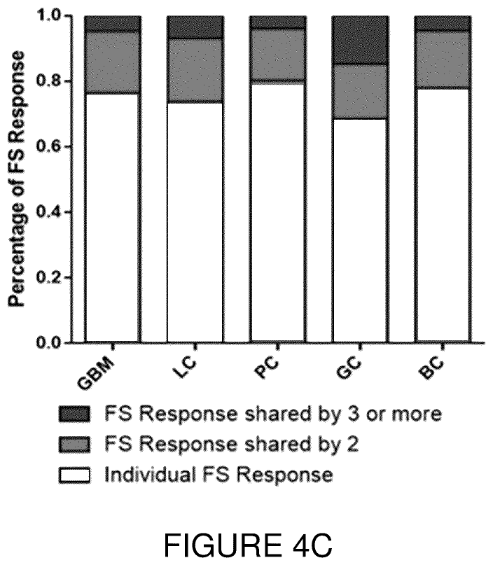

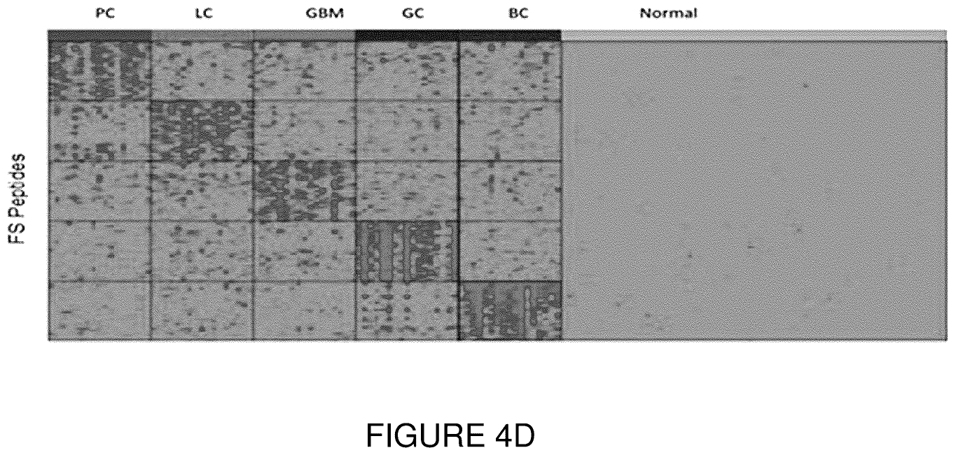

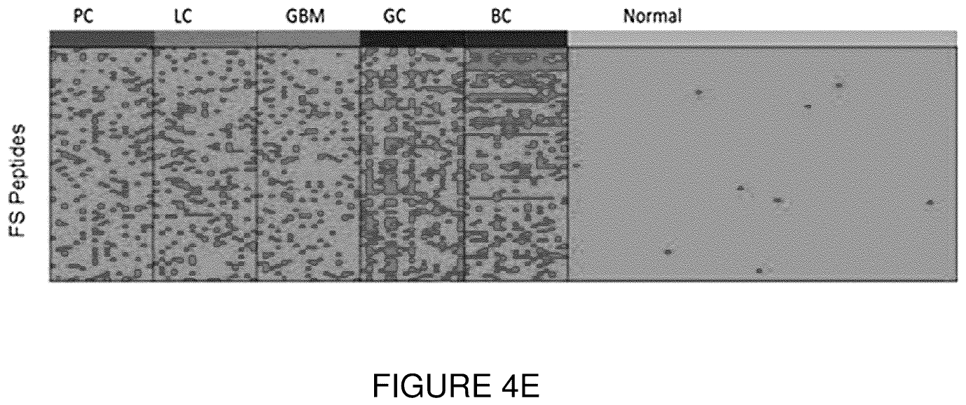

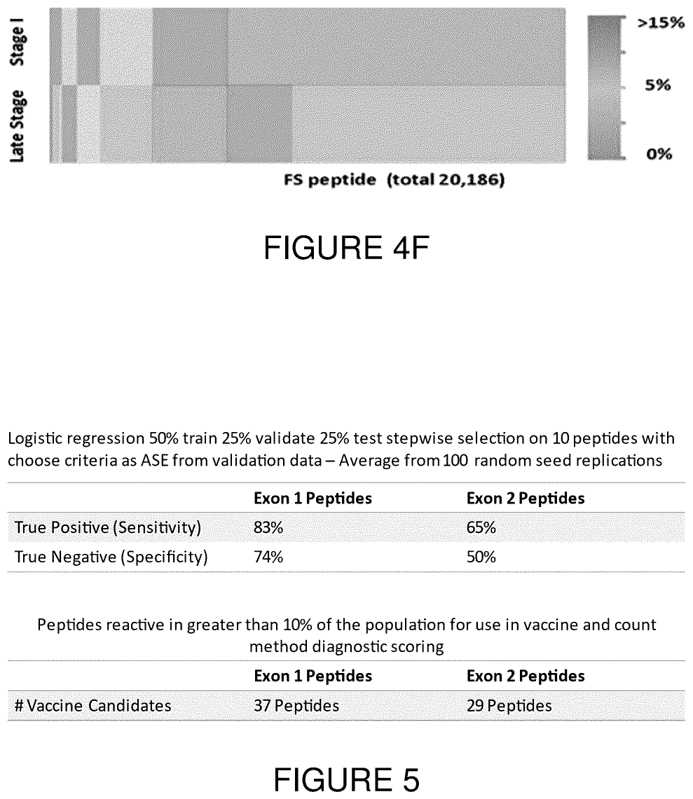

Each array was normalized to its median florescence for analysis. Three patterns of FS feature reactivity that were higher in cancer than non-cancer were found: common reactivity against FS peptides across all 5 cancer types; cancer type specific reactivity and personal reactivity. Reactivity against ˜7000 selected peptides are shown in FIG. 4A. Common reactivity and cancer type reactivity in 5 cancer types were marked with black squares. Non-cancer control samples had very low, sporadic reactivity in these FS peptides. As noted in FIG. 4A, there were also peptides that were commonly reactive in a cancer type. Based on this analysis a set of peptides could be chosen to optimize the number in common for a particular cancer. This is depicted in FIG. 4D for the 5 tumor types. The top 100 peptides based on the maximum coverage for the particular cancer type were chosen. These vaccine compositions are referred to herein as “focused” vaccines, as it is clear from the FIG. 4D that many of the peptides optimal for a particular cancer are shared across other cancer types.

Total reactivity on the 400K arrays was evaluated in the 5 cancer types and non-cancer samples with two methods. The first method compares the number of significant peptides in the cancer and control samples using fold change and p-values. By this method, BC, GC, PC and LC cancer samples had significantly more FS peptides compared to control samples which met the fold change and p-value criteria described in FIG. 4B. The exception is GBM where the reactivity in the controls was higher than the GBM samples. The second method used a scoring method for each FS peptide. A peptide is scored as positive if it is higher than six times the standard deviation (6SD) from the mean value of non-cancers for the peptide. All 5 cancer types had more positive FS peptides than the non-cancer controls.

The analysis of individual cancer samples within the same cancer type using the scoring method showed that there were three patterns of reactivity. Most of the positive FS peptides (about 69% to about 80%) were personal for that individual. However, about 16% to about 19% of the positive peptides were shared between two samples in that cancer type, with about 1.5% to about 6.9% shared between 3 or more. The distribution of these classes is shown in FIG. 4C. Gastric cancer samples had the highest shared FS response (6.9% were shared in 3 or more). This is consistent with the very high correlation coefficients in several gastric cancer samples.

In FIGS. 4D and 4E, people without cancer have sporadic antibody reactivity to some of the peptides. This has also been noted that healthy individuals have antibody and T-cell responses to tumor associated antigens. This could be due to random background cross-reactive IgG antibodies unrelated to cancer. It was previously shown that monoclonal antibodies are capable of binding random sequence peptides with high affinity, even though the peptides do not contain a sequence resembling the cognate site. Alternatively, this reactivity could be a manifestation of immune surveillance eliminating potential tumor cells. Any cell that produced and presented FS antigens, whether tumor or not, could be susceptible to this elimination, effectively a “bad cell” response.

Finally, it was determined if there were FS peptides that were common across all 5 cancer types that met the p-value and frequency requirements. In FIG. 4E, exemplary 100 candidate FS peptides for a pan-cancer (at least for the 5 considered) vaccine are presented. It has been found that there are extremely few recurrent mutations in the DNA of certain tumor types and with low chance of being immunogenic. In contrast common reactive FS variants can readily be identified.

All of the samples used for this analysis were from patients with late-stage cancer. Cancer vaccines could also potentially be used for treatment of early-stage cancers, and it is unclear whether early and late-stage cancer vaccines would require different components. 20,000 most reactive and recurrent peptides were compared to non-cancer for both the late stage and stage 1 pancreatic cancer. As evident in FIG. 4F, most of the peptides did not overlap between the late and early stages of pancreatic cancer. This implies that an early and late-stage vaccine would require distinct peptide compositions.

### Example 2: Exon 1 Frameshift Peptides

The array of all possible predicted RNA-defined frameshift peptides (FSP) was used in the analysis presented in FIG. 5. Analysis of the array revealed 19,997 FSP arising from mis-initiation of transcription at exon 1 encoded from the 5′ end of the RNA. These 19,997 FSP are referred to as Exon 1 FSP. The array contained 32,194 of FSP arising from a splicing error at exon 2 encoded from the 5′ end of the RNA. The 32,194 FSP are referred to as Exon 2 FSPs. The IgG signals of the array demonstrated that the level of IgG reactivity to Exon 1 FSP was equal to the level of IgG reactivity to Exon 2 FSP. The level of IgG reactivity to Exon 1 FSP was also equal to the level of IgG reactivity to Exon 3 FSP.

The unexpected result of robust antibody reactivity to Exon 1 FSP suggests the utility of Exon 1 FSP for use as cancer vaccines and/or cancer diagnostics. Exon 1 FSP are presumed to result from errors involving translation initiation in the ribosome. Translation mis-initiation in the ribosome gives the Exon 1 FSP and the associated antibodies a unique diagnostic profile compared to Exon 2 FSP, Exon 3 FSP, and their associated antibodies. For example, literature evidence suggests that cancerous tumors are correlated with an increased frequency of errors in translation initiation in the ribosome. Exon 1 FSP resulted in a 78% accuracy when predicting stage 1 BC compared to a 54% accuracy displayed by Exon 2 FSP in the same prediction. Exon 1 FSP includes 37 peptides that are reactive in 10% or more of patients having stage 1 BC. Exon 2 FSP includes 29 peptides that are reactive in 10% or more of patients having stage 1 BC. These FSP could serve as the basis of vaccines (FIG. 5). In FIG. 5, sera samples from women with stage 1 breast cancer were run on the FSP arrays. In the top panel, FSP from exon 1 and 2 were used to classify the positive and negative samples. Exon 1 peptides performed better than exon 2 peptides as diagnostic. In the bottom panel, reactive peptides that were so in more than 10% of the women with stage 1 breast cancer were chosen as vaccine candidates. More FSP met the requirements from exon 1 than exon 2.

### Example 3: Exon 1 FSPs to Predict Immunotherapy Response

Blood samples from 66 lung cancer patients undergoing immunotherapy were tested on a peptide array including Exon 1 frameshift peptides. Analysis of binding revealed 10,025 Exon 1 frameshift peptides that were bound by the lung cancer samples (SEQ ID NOs: 1-10,025). Among these, 32 were associated with immunotherapy treatment response. These Exon 1 frameshift peptides are presented in SEQ ID NOs: 1-32.

Samples were purchased from Indivmed, GmBH (Hamburg, Germany). The samples were obtained from patients malignant neoplasms of the upper lobe, middle lobe, bronchus or lung. Patients had undergone treatment with one or more checkpoint inhibitors including PD-1 inhibitors or PD-L1 inhibitors. Tumor response (including complete remission, partial remission, minimal response, stable disease, or progressive disease) had been noted for each sample.

Samples were assayed on a high-density peptide microarray. We designed and produced theses high-density, in situ-synthesized peptide microarrays that display informatically predicted FSPs. Each array contains 374,082 15-mer peptides corresponding to 190,865 predicted frameshift neoantigens that could be generated from i) exon splicing errors, ii) exon 1 translational mis-initiation or iii) transcriptional slippage within microsatellite regions. There are 34,733 exon 1 mis-initiation FSPs on the array.

Binding of antibodies within the serum samples to the peptides on the array was measured quantitatively. Specifically, serum samples were diluted 1:50 into an incubation buffer (0.75% casein in phosphate buffered saline with 0.25% tween20, PBST). The diluted sera samples (200 ml) were incubated in individual arrays using a gasketed cassette at room temperature for 24 hours. Following 3 washes with 1× PBST, peptide bound antibodies were detected by incubation with 4 nM of Dylight 550 labeled goat anti-human Fc IgG secondary antibody (ThermoFisher Scientific, Cat #SA5-10135) in 0.75% casein/PBST at 37° C. for 1 hr. Slides were washed 3 times with 1× PBST, twice with dH₂O and once each with 40% and 100% isopropanol. Slides were dried by centrifuging at 800 RPM for 2 minutes. The fluorescent signal of bound secondary antibody was detected in an InnoScan 910 laser scanner (Innopsys, France) and the raw relative fluorescent units (RFU) were extracted and tabulated using MAPIX (Innopsys, France) gridding software.

Analysis via a chi-square test identified 226 peptides which significantly differentiated samples based on immunotherapy response to treatment. Of these 226 frameshift peptides, 32 were Exon 1 frameshift peptides. At least one of these 32 Exon 1 frameshift peptides are significantly reactive in 23 of the samples; two or more of these Exon 1 frameshift peptides are reactive in 15 samples. Using standard contrast scoring, every sample which demonstrated reactivity on two or more of the 32 Exon 1 frameshift peptides was correctly classified. However, 34 of the 66 samples showed less than 2 reactive FSPS. These samples were considered indeterminate. Removing the 32 Exon 1 frameshift peptides from the whole 226 model causes an increase in the indeterminate rate by 7.6%, indicating that the exon 1 FSPs provided information for classifying the patient samples.

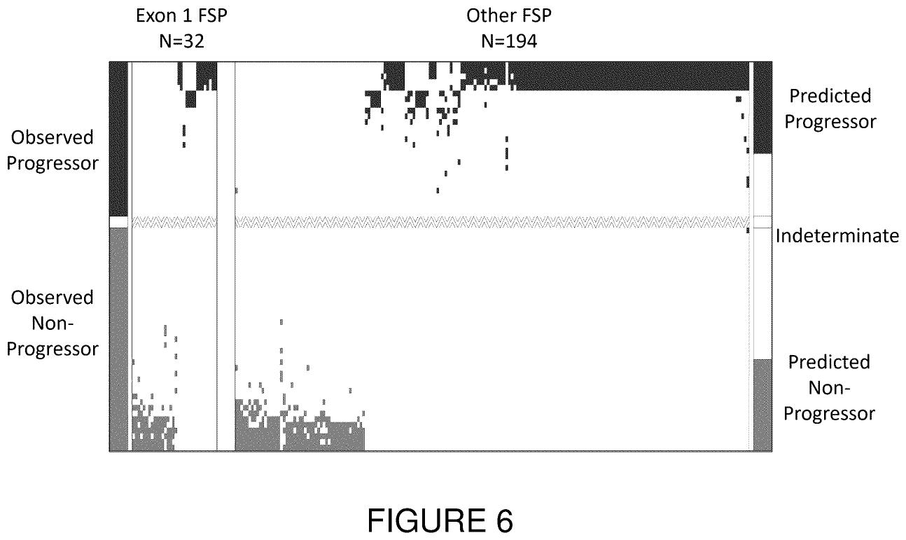

Results are presented in FIG. 6. The exon 1 FSP response to treatment prediction model is compared to observed response outcomes. A heat map representation of observed or predicted responders (bottom) and observed or predicted non-responders (top) was prepared to visualize the model performance.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

All publications, patents, and patent applications mentioned in this specification are herein incorporated by reference to the same extent as if each individual publication, patent, or patent application was specifically and individually indicated to be incorporated by reference.

What is claimed is:

1. An array comprising frameshift peptides (FSPs) resulting from exon 1 mis-initiation of translation.

2. The array of claim 1, wherein the FSPs on the array are spaced between 3 and 9 μm.

3. The array of claim 1, wherein the FSPs comprise one or more peptides as set forth in any one of SEQ ID NOs: 1-19,997.

4. The array of claim 1, wherein the array comprises at least about 2500, about 5000, about 7500, about 10000, about 12500, about 15000, about 17500, about 20000, about 22500, about 25000, about 27500, about 30000, about 32500, about 35000, about 37500, about 40000, about 50000, about 100000, about 200000, about 300000, or about 400000 FSPs.

5. A vaccine composition comprising frameshift peptides (FSPs) resulting from exon 1 mis-initiation of translation.

6. The vaccine composition of claim 5, further comprising an adjuvant.

7. The vaccine composition of claim 6, wherein the adjuvant is ABM2, AS01B, AS02, AS02A, Adjumer, Adjuvax, Algammulin, Alum, Aluminum phosphate, Aluminum potassium sulfate, Bordetella pertussis, Calcitriol, Chitosan, Cholera toxin, CpG, Dibutyl phthalate, Dimethyldioctadecylammonium bromide (DDA), Freund's adjuvant, Freund's complete, Freund's incomplete (IFA), GM-CSF, GMDP, Gamma Inulin, Glycerol, HBSS (Hank's Balanced Salt Solution), Hiltonol, IL-12, IL-2, Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non-ionic copolymer), Pam3Cys (lipoprotein), Pertussis toxin, Poloxamer, QS21, RaLPS, Ribi, Saponin, Seppic ISA 720, Soybean Oil, Squalene, Syntex Adjuvant Formulation (SAF), Synthetic polynucleotides (poly IC/poly AU), TiterMax Tomatine, Vaxfectin, XtendIII, or Zymosan.

8. A method of detecting cancer in a subject, the method comprising:
contacting a biological sample from the subject to an array comprising frameshift peptides (FSPs) resulting from exon 1 mis-initiation of translation; and
measuring antibody reactivity to the FSPs resulting from mis-initiation of translation in tumors.

9. The method of claim 8, wherein the FSPs comprise one or more peptides as set forth in any one of SEQ ID NOs: 1-19,997.

10. The method of claim 8, wherein the FSPs are fixed on a substrate.

11. The method of claim 10, wherein the substrate comprises glass, silica, composite, resin, or combination thereof.

12. The method of claim 8, wherein the array is configured to detect binding by at least one of fluorescence, luminescence, calorimetry, chromatography, radioactivity, Bio-Layer Interferometry, and surface plasmon resonance.

13. The method of claim 8, wherein the array comprises at least about 2500, about 5000, about 7500, about 10000, about 12500, about 15000, about 17500, about 20000, about 22500, about 25000, about 27500, about 30000, about 32500, about 35000, about 37500, about 40000, about 50000, about 100000, about 200000, about 300000, or about 400000 FSPs.

14. The method of claim 8, wherein the biological sample comprises blood, serum, plasma, cerebrospinal fluid, saliva, urine, or combinations thereof.

15. The method of claim 8, wherein the biological sample comprises an antibody.

16. The method of claim 8, wherein the subject is a mammal.

17. A method of treating or preventing cancer, comprising administering a composition comprising: a therapeutic molecule designed to bind frameshift peptides (FSPs) resulting from exon 1 mis-initiation of translation, or FSPs resulting from exon 1 mis-initiation of translation.

18. The method of claim 17, wherein the FSPs comprise one or more peptides as set forth in SEQ ID NOs: 1-19,997.

19. A method of predicting response to an immunotherapy, comprising measuring antibody reactivity to one or more frameshift peptides (FSPs) resulting from exon 1 mis-initiation of translation.

20. The method of claim 19, wherein the one or more FSPs comprise one or more peptides as set forth in any one of SEQ ID NOs: 1-19,997.
